(19) United States
(12) Patent Application Publication
(10) Pub. No.: US 2020/0375911 A1
(43) Pub. Date: Dec. 3, 2020

(54) CANNABIDIOL NANOCRYSTAL COMPOSITIONS

(71) Applicant: Fresh Cut Development, LLC, Chandler, AZ (US)

(72) Inventors: Goutham R. Adelli, Tempe, AZ (US); Thromoorthy Potta, Phoenix, AZ (US); Venkat Goskonda, Phoenix, AZ (US)

(21) Appl. No.: 16/874,300

(22) Filed: May 14, 2020

Related U.S. Application Data
(60) Provisional application No. 62/856,526, filed on Jun. 3, 2019.

(51) Int. Cl. A61K 9/51; A61K 31/352; A61K 47/38; A61K 47/26; A61K 47/22; A61K 47/10; A61K 47/14

(52) U.S. Cl. CPC A61K 9/51; A61K 31/352; A61K 47/38; A61K 47/14; A61K 47/22; A61K 47/10; A61K 47/26

(57) ABSTRACT

The present invention is directed to a nanocrystal cannabidiol composition containing one or more lipids and one or more stabilizers. The present invention is further directed to a process of preparing a nanocrystal cannabidiol composition. The present invention is further directed to a method of treating a disease comprising administering a composition of the present invention to a subject in need thereof. The present invention is further directed to a method of treating withdrawal symptoms comprising administering a composition of the present invention to a subject in need thereof.

### CROSS REFERENCE TO RELATED APPLICATION

This application claims priority to U.S. application No. 62/856,526 filed Jun. 3, 2019 (expired). The entire contents are which are hereby incorporated herein by reference in their entirety.

### FIELD OF THE INVENTION

The present invention is directed to a nanocrystal cannabidiol composition containing one or more lipids and one or more stabilizers. The present invention is further directed to a process of preparing a nanocrystal cannabidiol composition. The present invention is further directed to a method of treating a disease comprising administering a composition of the present invention to a subject in need thereof. The present invention is further directed to a method of treating withdrawal symptoms comprising administering a composition of the present invention to a subject in need thereof.

### BACKGROUND OF THE INVENTION

Cannabidiol, (−)-trans-2-p-mentha-1,8-dien-3-yl-5-pentylresorcinol, is non-psychoactive and has shown promise in treating numerous diseases and disorders. Cannabidiol has been approved by the United States Food and Drug Administration for to treat Lennox-Gastaut syndrome, Dravet syndrome. Further, cannabidiol, may be suitable for the treatment of diseases or disorders, or symptoms of diseases or disorders, such as mycolonic seizures, juvenile mycolonic epilepsy, refractory epilepsy, schizophrenia, juvenile spasms, West syndrome, refractory infantile spasms, infantile spasms, tubular sclerosis complex, brain tumors, neuropathic pain, cannabis use disorder, post-traumatic stress disorder, anxiety, early psychosis, Alzheimer's Disease autism, and withdrawal from opioids, cocaine, heroin, amphetamines, and nicotine.

While there are many dosage forms of cannabidiol the most popular form is oral. Oral formulations of cannabidiol are more convenient, and are more likely to lead to patient compliance. Oral dosages of cannabidiol has been formulated in hydroalcoholic and lipid-based formulations. The issue with these oral formulations is that they have poor solubility and thus poor bioavailability in water such as encountered in the gastrointestinal tract when imbibed.

To combat poor solubility formulation scientist have developed nanocrystal formulations. Nanocrystal formulations have shown to improve solubilization of poorly soluble drugs, improve the bioavailability due to reduced first pass metabolism and improved absorption through lymphatic transport by forming chylomicrons. However, developing a nanocrystal formulation is a painstaking task that differs for each active ingredient. The specific excipients and concentrations may only be discovered through intense formulation research.

Accordingly, there is a need in the art for a cannabidiol formulation that forms nanocrystals.

### SUMMARY OF THE INVENTION

The present invention is directed to a nanocrystal composition comprising from about 1% to about 40% w/w cannabidiol, one or more lipids selected from stearoyl polyoxyl-32 glycerides, polyethylene glycol monostearate, glyceryl dibehenate, glyceryl distearate, propylene glycol monocaprylate, oleoyl polyoxyl-6 glycerides and linoleoyl polyoxyl-6 glycerides and one or more stabilizers selected from hydroxypropyl cellulose, hydroxypropyl methyl cellulose, polyvinyl pyrrolidine and a poloxamer.

The present invention is further directed to a nanocrystal composition comprising:
from about 5% to about 20% w/w cannabidiol:
from about 0.1% to about 5% w/w of one or more lipids selected from stearoyl polyoxyl-32 glycerides, polyethylene glycol monostearate, glyceryl dibehenate, glyceryl distearate, propylene glycol monocaprylate, oleoyl polyoxyl-6 glycerides and linoleoyl polyoxyl-6 glycerides; and
from about 1% to about 10% w/w of one or more stabilizers selected from hydroxypropyl cellulose, hydroxypropyl methyl cellulose, polyvinyl pyrrolidine and a poloxamer.

The present invention is further directed to a process of producing a nanoparticle composition comprising the steps of
adding ascorbic acid or vitamin E pegylated, polysorbate 80, poloxamer 188. hydroxypropyl cellulose L and optionally, EDTA disodium to water were while stirring to create an aqueous phase;
adding cannabidiol to ethanol while stirring to create an alcohol phase;
adding the alcohol phase to the aqueous phase dropwise while spinning in a homogenizer at from about 13,000 to about 17,000 revolutions per minute for 5 minutes to form a coarse mixture;
placing the coarse mixture in a pressure homogenizer for from about 5 to about 10 cycles at from about 10,000 to about 20,000 pounds per square inch to create a homogenous mixture; and
allowing the homogenous mixture to reach room temperature,
   wherein the process provides a particle size range from about 200 to about 500 nanometers.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures.

FIG. 1. Shows an illustration of plasma concentration of cannabidiol after administration of Composition 1 from time 0 to 96 hours.

FIG. 2. Shows an illustration of plasma concentration of cannabidiol after administration of Composition 1 from time 0 to 8 hours.

### DETAILED DESCRIPTION OF THE INVENTION

The detailed description of exemplary embodiments herein makes reference to the accompanying drawings which show exemplary embodiments by way of illustration and their best mode. While these exemplary embodiments are described in sufficient detail to enable those skilled in the art to practice the inventions, it should be understood that other embodiments may be realized and that logical, chemical, and mechanical changes may be made without departing from the spirit and scope of the inventions. Thus, the detailed description, herein is presented for purposes of illustration only and not of limitation. For example, the steps herein recited in any of the method of process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Applicant unexpectedly found that the presence of particular excipients in a cannabidiol composition form nanocrystals capable of passing through the gastrointestinal tract.

In one embodiment, the present invention is directed to a nanocrystal composition comprising from about 1% to about 40% w/w cannabidiol, one or more lipids selected from stearoyl polyoxyl-32 glycerides, polyethylene glycol monostearate, glyceryl dibehenate, glyceryl distearate, propylene glycol monocaprylate, oleoyl polyoxyl-6 glycerides and linoleoyl polyoxyl-6 glycerides and one or more stabilizers selected from hydroxypropyl cellulose, hydroxypropyl methyl cellulose, polyvinyl pyrrolidine and a poloxamer.

Cannabidiol may be present in compositions of the present invention at a concentration from about 0.1% to about 50% w/w, preferably from about 1% to about 40% w/w, more preferably from about 5% to about 20% w/w and even more preferably from about 5% to about 10% w/w.

Lipids suitable for use in compositions of the present invention include, but are not limited to, stearoyl polyoxyl-32 glycerides (Gelucire®50/13), polyethylene glycol monostearate (Gelucire® 48/16), glyceryl dibehenate (Compritol® 888 ATO). glyceryl distearate (Precirol® ATO 5), propylene glycol monocaprylate (Caproyl® 90), oleoyl polyoxyl-6 glycerides (Labrafil® M 1944 CS) and linoleoyl polyoxyl-6 glycerides (Labrafil® M 2125 CS). In a preferred embodiment, lipids may be selected from stearoyl polyoxyl-32 glycerides, oleoyl polyoxyl-6 glycerides, linoleoyl polyoxyl-6 glycerides and a combination thereof.

The one or more lipids may be present in the compositions of the present invention at a concentration from about 0.1% to about 10% w/w, preferably from about 0.1% to about 5% w/w and even more preferably from about 0.3% to about 1% w/w.

Stabilizers suitable for use in the present invention include, but are not limited to, hydroxypropyl cellulose, hydroxypropyl methyl cellulose, polyvinyl pyrrolidine and a poloxamer. In a preferred embodiment the stabilizer is hydroxypropyl cellulose L.

The one or more stabilizers may be present in the compositions of the present invention at a concentration from about 1% to about 10% w/w, preferably from about 1% to about 5% w/w and even more preferably from about 2% to about 4% w/w.

Surfactants suitable for use in the present invention include, but are not limited to, cetyl trimethylammonium bromide (CTAB) and cetyl trimethylammonium chloride (CTAC), ammonium lauryl sulfate, sodium lauryl sulfate, polysorbate 20, polysorbate 40, polysorbate 60, polysorbate 80, sorbitan monolaurate (Span® 20), sorbitan monopalmitate (Span® 40), sorbitan monostearate (Span® 60), and sorbitan monooleate (Span® 80), poloxamer 188 and poloxamer 407. In a preferred embodiment, surfactants are selected from polysorbate 80, sorbitan monooleate, poloxamer 188 and a combination thereof.

The one or more surfactants may be present in the compositions of the present invention at a concentration from about 1% to about 10% w/w, preferably from about 1% to about 7.5% w/w and even more preferably from about 3% to about 7.5% w/w.

Cosolvents suitable for use in the present invention include, but are not limited to, water, propylene glycol, polyethylene glycol 400, polyethylene glycol 600, polyethylene glycol 1000, glycerol, isopropyl alcohol, sesame oil, olive oil, ethanol or a combination thereof. In a preferred embodiment, the cosolvent is a combination of water and ethanol.

The one or more cosolvents may be present in compositions of the present invention at a concentration from about 50% to about 90% w/w, preferably from about 70% to about 90% w/w and even more preferably from about 77% to about 88% w/w.

Ethanol may be present in compositions of the present invention at a concentration from about 1% to about 20% w/w, preferably from about 5% to about 20% w/w and even more preferably from about 7.5% to about 15% w/w.

Water may be present in compositions of the present invention at a concentration from about 40% to about 90% w/w, preferably from about 60% to about 80% w/w and even more preferably from about 62% to about 80% w/w.

Preservatives suitable for use in the present invention include, but are not limited to, meta-cresol, benzalkonium chloride, methyl paraben and propyl paraben.

Antioxidants suitable for use in the present invention include, but are not limited to, pegylated alpha-tocopherol isomer of vitamin E, alpha-tocopherol, ascorbic acid, ascorbyl palmitate, butylated hydroxyanisole, butylated hydroxytoluene and combination thereof. In a preferred embodiment, the preservative is selected from pegylated alpha-tocopherol isomer of vitamin E, ascorbic acid and a combination thereof.

The one or more preservatives may be present in compositions of the present invention at a concentration from about 0.1% to about 5% w/w, preferably from about 1% to about 5% w/w and even more preferably from about 1% to about 2% w/w.

Disodium edetate (“EDTA disodium”) may be present in compositions of the present invention at a concentration from about 0.01% to about 0.5% w/w, preferably about 0.1% w/w.

In a preferred embodiment, the one or more surfactants is selected from the group of polysorbate 80, sorbitan monooleate and poloxamer 188, wherein the ratio of polysorbate 80 or sorbitan monooleate to poloxamer 188 is about 2:1.

In another preferred embodiment, the one or more stabilizers is hydroxypropyl cellulose L and wherein the ratio of cannabidiol to hydroxypropyl cellulose L is about 2:1.

In another preferred embodiment, the compositions of the present invention forms particles having a mean particle size from about 100 to about 1000 nanometers, more preferably from about 200 to about 500 nanometers and even more preferably from about 250 to about 300 nanometers.

In a preferred embodiment, the present invention is directed to a nanocrystal composition comprising from about 5% to about 20% w/w cannabidiol;
from about 0.1% to about 5% w/w of one or more lipids selected from stearoyl polyoxyl-32 glycerides, polyethylene glycol monostearate, glyceryl dibehenate, glyceryl distearate, propylene glycol monocaprylate, oleoyl polyoxyl-6 glycerides and linoleoyl polyoxyl-6 glycerides;
from about 1% to about 10% w/w of one or more stabilizers selected from hydroxypropyl cellulose, hyrdroxypropyl methyl cellulose, polyvinyl pyrrolidine and a poloxamer;
optionally, from about 1% to about 10% w/w of one or more surfactants selected from polysorbate 20, polysorbate 40, polysorbate 60, polysorbate 80, sorbitan monolaurate, sorbitan monopalmitate, sorbitan monostearate, and sorbitan monooleate, poloxamer 188 and poloxamer 407;
optionally, from about 50% to about 90% w/w of one or more cosolvents selected from water, propylene glycol and ethanol;
optionally, from about 0.1% to about 5% w/w of one or more preservatives selected from pegylated alpha-tocopherol isomer of vitamin, alpha-tocopherol, ascorbic acid, ascorbyl palmitate, methyl paraben and propyl paraben; and
optionally, from about 0.01% to about 0.5% w/w disodium edetate.

In a more preferred embodiment, the present invention is directed to a nanoparticle composition comprising:
from about 5.49% w/w to about 10.98% w/w cannabidiol;
from about 7.5% to about 15.0% w/w ethanol;
about 2.63% w/w hydroxypropyl cellulose L;
about 1.0% to about 5.0% w/w polysorbate 80; and
from about 0.5% to about 2.5% w/w sorbitan monooleate, poloxamer 188 or a combination thereof

In another embodiment. the present invention is directed to a process of producing a nanoparticle composition comprising the steps of:
adding ascorbic acid or vitamin E pegylated, polysorbate 80, poloxamer 188, hydroxypropyl cellulose L and optionally, EDTA disodium to water were while stirring to create an aqueous phase;
adding cannabidiol to ethanol while stirring to create an alcohol phase;
adding the alcohol phase to the aqueous phase dropwise while spinning in a homogenizer at from about 13,000 to about 17,000 revolutions per minute for 5 minutes to form a coarse mixture;
placing the coarse mixture in a pressure homogenizer for from about 5 to about 10 cycles at from about 10,000 to about 20,000 pounds per square inch to create a homogenous mixture; and
allowing the homogenous mixture to reach room temperature, wherein the process provides a particle size range from about 200 to about 500 nanometers.

In another embodiment, the present invention is directed to a method of treating a disease selected from Prader-Willi syndrome, obesity, graft versus host disease, gelastic seizures/hypothalamic hamartoma, neonatal seizures, dystonia, central pain syndromes, phantom limb pain, multiple sclerosis, traumatic brain injury, radiation therapy, acute graft versus host disease, chronic graft versus host disease, T-cell autoimmune disorders, colitis, Dravet Syndrome, Lennox Gastaut Syndrome, mycolonic seizures, juvenile mycolonic epilepsy, refractory epilepsy, childhood absence epilepsy, schizophrenia, juvenile spasms, West syndrome, infantile spasms, refractory infantile spasms, tuberous sclerosis complex, brain tumors, neuropathic pain, cannabis use disorder, post-traumatic stress disorder, anxiety, early psychosis, Alzheimer's Disease, autism, acne, Parkinson's disease, social anxiety disorder. depression, diabetic retinopathy, diabetic nephropathy, diabetic neuropathy, ischemic injury of heart, ischemic injury of brain, chronic pain syndrome, and rheumatoid arthritis comprising administering a composition of the present invention to a subject in need thereof.

In another embodiment, the present invention is directed to a method of treating withdrawal symptoms comprising administering a composition of the present invention to a subject in need thereof, wherein the withdrawal symptoms are caused by the subject reducing or quitting use of an opioid, cocaine, heroin, an amphetamine or nicotine.

As used herein, all numerical values, relating to amounts, weights, and the like, that are defined as “about” each particular value is plus or minus 10%. For example, the phrase “about 10% w/w” is to be understood as “9% w/w to 11% w/w.” Therefore, amounts within 10% of the claimed value are encompassed by the scope of the claims.

As used herein “% w/w” and “percent w/w” refer to the percent weight of the total formulation.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

### EXAMPLES

### Example 1

### Preparation of Compositions

TABLE 1
Compositions of the Invention

| % w/w | 1 | 2 | 3 | 4 | 5 |
|---|---|---|---|---|---|
| Cannabidiol | 5.490 | 10.980 | 5.490 | 5.490 | 5.490 |
| Ethanol | 7.500 | 15.000 | 7.500 | 7.500 | 7.500 |
| Stearoyl polyoxyl-32 | — | — | — | — | 0.375 |
| glycerides | | | | | |
| Oleoyl polyoxyl-6 | — | — | 1.000 | — | — |
| glycerides | | | | | |
| Linoleoyl polyoxyl-6 | — | — | — | 0.536 | — |
| glycerides | | | | | |
| Hydroxypropyl | 2.633 | 2.633 | 2.633 | 2.633 | 2.633 |
| cellulose L | | | | | |
| Polysorbate 80 | 2.500 | 5.000 | 2.500 | 2.500 | 1.000 |
| Poloxamer 188 | 1.250 | — | 1.250 | 1.250 | 0.500 |
| Sorbitan monooleate | — | 2.500 | 0.800 | 0.800 | 1.500 |
| Pegylated vitamin E | — | 1.334 | 1.334 | 1.334 | 1.334 |
| Ascorbic acid | 1.000 | — | — | — | — |
| EDTA disodium | 0.100 | — | — | — | — |
| Water | 79.527 | 62.553 | 77.493 | 77.957 | 79.668 |

Gelucire® 50/13 was used as the source of stearoyl polyoxyl-32 glycerides and is a registered trademark of and available from Gattefosse SAS.

Labrafil® M 1944 CS was used as the source of oleoyl polyoxyl-6 glycerides and is a registered trademark of and available from Gattefosse SAS.

Labrafil® M 2125 CS was used as the source of linoleoyl polyoxyl-6 glycerides and is a registered trademark of and available from Gattefosse SAS.

Span® 80 was used as the source of sorbitan monooleate and is a registered trademark of and available from Uniqema Americas LLC.

### Method

Water, ascorbic acid or vitamin E pegylated, polysorbate 80, poloxamer 188, hydroxypropyl cellulose L and optionally, EDTA disodium were mixed while stirring to create an aqueous phase. Ethanol and cannabidiol were mixed to create an alcohol phase. Once dissolved, the alcohol phase is added to the aqueous phase dropwise in an Ultra-Turrax homogenizer for 5 minutes at 13,000 to 17,000 rpm to form a coarse mixture. The course mixture was then transferred to an Avestin C5 emulsiflex high pressure homogenizer and run for 5 to 10 cycles at 10,000 to 20.000 pounds per square inch to create a homogenous mixture. The homogenous mixture was then collected and allowed to reach room temperature. Particle size distribution of the compositions were measured using a Nicomp nano ZS.

### Results

Particle size range of the compositions were from 250 to 500 nanometers.

### Example 2

### Stability of Compositions of the Invention

### Methods

Compositions 1 and 2 of Table 1, above, were prepared as in Example 1, above and subjected to 40° C.±2° C. and 75±5% relative humidity (“RH”) for 3 months and/or 25° C.±2° C. and 60±5% RH for 3 months and/or 5° C.±2° C. for 3 months. Results can be seen in Tables 2-5, below.

TABLE 2
Stability of Composition 1

| | RRT | Specification | T = 0 | 2 wk 40 C. | 4 wk 40 C. | 8 wk 40 C. | 3 M 40 C. |
|---|---|---|---|---|---|---|---|
| Physical appearance | | | White | Pale Yellow | Pale Yellow | Creamy white | Light |
| | | | Suspension | Suspension | Suspension | Suspension | creamy brown |
| | | | | | | | Suspension |
| Assay (% of Label Claim of 50 mg/mL) | | | 101.33 | 100.97 | 98.24 | 107.85 | 103.47 |
| Cannabinol | 1.43 | ≤0.15% | ND | ND | ND | ND | ND |
| Cis-cannabidiol | 1.53 | ≤0.15% | ND | ND | ND | ND | ND |
| Delta 9-THC | 1.83 | ≤0.15% | ND | ND | ND | 0.02% | ND |
| Trans-(1R, 6R)-3′- | 1.97 | ≤0.15% | 0.04% | 0.04% | 0.03% | 0.04% | 0.03% |
| methyl-cannabidiol | | | | | | | |
| Delta 8-THC | 2.16 | ≤0.15% | ND | ND | ND | 0.03% | ND |
| Unknown Impurities | 0.35 | ≤0.15% | ND | ND | ND | ND | ND |
| | 0.37 | ≤0.15% | ND | ND | ND | ND | 0.04% |
| | 0.44 | ≤0.15% | ND | 0.02% | ND | ND | ND |
| | 0.46 | ≤0.15% | ND | ND | 0.04% | 0.08% | ND |
| | 0.47 | ≤0.15% | ND | ND | ND | ND | 0.12% |
| | 0.53 | ≤0.15% | ND | ND | 0.03% | ND | ND |
| | 0.55 | ≤0.15% | ND | 0.02% | ND | 0.08% | 0.06% |
| | 0.75 | ≤0.15% | 0.04% | 0.04% | 0.04% | 0.04% | 0.11% |
| | 1.10 | ≤0.15% | 0.02% | 0.02% | ND | ND | ND |
| | 1.12 | ≤0.15% | ND | ND | 0.04% | ND | ND |
| | 1.14 | ≤0.15% | 0.02% | ND | ND | 0.09% | 0.08% |
| | 1.17 | ≤0.15% | ND | ND | 0.01% | ND | ND |
| | 1.33 | ≤0.15% | ND | ND | ND | ND | 0.02% |
| | 1.81 | ≤0.15% | ND | ND | ND | ND | 0.03% |
| | 2.17 | ≤0.15% | ND | ND | ND | ND | 0.05% |
| | 2.67 | ≤0.15% | ND | ND | ND | ND | 0.02% |
| | 2.93 | ≤0.15% | ND | ND | ND | ND | 0.03% |
| | 3.48 | ≤0.15% | ND | ND | ND | ND | 0.24% |

| | | | | | | |
|---|---|---|---|---|---|---|
| Total Impurities | ≤5.0% | 0.12% | 0.14% | 0.19% | 0.38% | 0.83% |

TABLE 3
Stability of Composition I

| | RRT | Specification | T = 0 | 4 wk 25 C. | 8 wk 25 C. | 3 M 25 C. |
|---|---|---|---|---|---|---|
| Physical appearance | | | White | White | Milky white | Creamy white |
| | | | Suspension | suspension | Suspension | Suspension |
| Assay (% of Label Claim of 50 mg/mL) | | | 101.33 | 97.90 | 107.63 | 107.45 |
| Cannabinol | 1.43 | ≤0.15% | ND | ND | ND | ND |
| Cis-cannabidiol | 1.53 | ≤0.15% | ND | ND | ND | ND |
| Delta 9-THC | 1.83 | ≤0.15% | ND | ND | ND | ND |
| Trans-(1R, 6R)-3′- | 1.97 | ≤0.15% | 0.04% | 0.03% | 0.04% | 0.04% |
| methyl-cannabidiol | | | | | | |
| Delta 8-THC | 2.16 | ≤0.15% | ND | ND | ND | ND |
| Unknown impurities | 0.46 | ≤0.15% | ND | 0.03% | 0.03% | ND |
| | 0.47 | ≤0.15% | ND | ND | ND | 0.04% |
| | 0.53 | ≤0.15% | ND | 0.01% | ND | ND |
| | 0.55 | ≤0.15% | ND | ND | 0.04% | 0.09% |
| | 0.75 | ≤0.15% | 0.04% | 0.04% | 0.04% | 0.08% |
| | 0.83 | ≤0.15% | ND | ND | 0.01% | ND |
| | 1.10 | ≤0.15% | 0.02% | ND | ND | ND |
| | 1.12 | ≤0.15% | ND | 0.02% | ND | ND |
| | 1.14 | ≤0.15% | 0.02% | ND | 0.04% | 0.03% |
| | 1.17 | ≤0.15% | ND | 0.01% | ND | ND |
| | 2.67 | ≤0.15% | ND | ND | ND | 0.02% |
| | 2.93 | ≤0.15% | ND | ND | ND | 0.02% |
| | 3.48 | ≤0.15% | ND | ND | ND | 0.02% |

| | | | | | |
|---|---|---|---|---|---|
| Total Impurities | ≤5.0% | 0.12% | 0.14% | 0.20% | 0.34% |

TABLE 4
Stability of Composition 2

| | RRT | Specification | T = 0 | 2 wk 40 C. | 4 wk 40 C. | 8 wk 40 C. |
|---|---|---|---|---|---|---|
| Physical appearance | | | Milky white | Milky white | Creamy white | Light brown |
| | | | suspension | suspension | suspension | suspension |
| Assay (% of Label Claim of 50 mg/mL) | | | 107.66 | 107.61 | 107.38 | 101.45 |
| Cannabinol | 1.43 | ≤0.15% | ND | ND | ND | 0.01% |
| Cis-cannabidiol | 1.53 | ≤0.15% | ND | ND | ND | 0.04% |
| Delta 9-THC | 1.83 | ≤0.15% | ND | ND | ND | ND |
| Trans-(1R, 6R)-3′- | 1.97 | ≤0.15% | 0.03% | 0.03% | 0.05% | 0.10% |
| methyl-cannabidiol | | | | | | |
| Delta 8-THC | 2.16 | ≤0.15% | ND | ND | ND | 0.04% |
| Unknown impurities | 0.37 | ≤0.15% | ND | ND | 0.07% | ND |
| | 0.43 | ≤0.15% | ND | ND | 0.06% | 0.08% |
| | 0.46 | ≤0.15% | 0.03% | 0.19% | 0.48% | 1.15% |
| | 0.57 | ≤0.15% | ND | ND | 0.03% | 0.07% |
| | 0.61 | ≤0.15% | ND | ND | ND | 0.01% |
| | 0.65 | ≤0.15% | ND | ND | ND | 0.03% |
| | 0.68 | ≤0.15% | ND | ND | ND | 0.03% |
| | 0.76 | ≤0.15% | 0.05% | 0.18% | 0.52% | 1.47% |
| | 0.82 | ≤0.15% | 0.01% | 0.05% | 0.15% | 0.24% |
| | 0.90 | ≤0.15% | ND | ND | ND | 0.05% |
| | 1.14 | ≤0.15% | ND | ND | 0.02% | 0.04% |
| | 1.33 | ≤0.15% | ND | ND | 0.05% | 0.10% |
| | 1.35 | ≤0.15% | 0.01% | ND | ND | ND |
| | 2.46 | ≤0.15% | ND | ND | ND | 0.04% |
| | 3.28 | ≤0.15% | ND | ND | 0.03% | ND |

| | | | | | |
|---|---|---|---|---|---|
| Total Impurities | ≤5.0% | 0.13% | 0.45% | 1.46% | 3.50% |

TABLE 5
Stability of Composition 2

| Components | RRT | Spec. | T = 0 | 4 wk 25 C. | 8 wk 25 C. | 3 M 25 C. | 3 M 5 C. |
|---|---|---|---|---|---|---|---|
| Physical appearance | | | Milky white | Milky white | Creamy white | Creamy white | Milky white |
| | | | suspension | suspension | suspension | suspension | suspension |
| Assay (% of Label Claim of 50 mg/mL) | | | 107.66 | 109.48 | 108.61 | 107.54 | 110.01 |
| Cannabinol | 1.43 | ≤0.15% | ND | ND | ND | ND | ND |
| Cis-cannabidiol | 1.53 | ≤0.15% | ND | ND | ND | ND | ND |
| Delta 9-THC | 1.83 | ≤0.15% | ND | ND | ND | ND | ND |
| Trans-(1R, 6R)-3′- | 1.97 | ≤0.15% | 0.03% | 0.04% | 0.03% | 0.04% | 0.04% |
| methyl-cannabidiol | | | | | | | |
| Delta 8-THC | 2.16 | ≤0.15% | ND | ND | ND | ND | ND |
| Unknown impurities | 0.37 | ≤0.15% | ND | ND | ND | 0.03% | ND |
| | 0.43 | ≤0.15% | ND | ND | 0.02% | 0.04% | ND |
| | 0.46 | ≤0.15% | 0.03% | 0.05% | 0.11% | 0.21% | 0.06% |
| | 0.57 | ≤0.15% | ND | ND | 0.01% | ND | ND |
| | 0.76 | ≤0.15% | 0.05% | 0.08% | 0.22% | 0.26% | 0.07% |
| | 0.82 | ≤0.15% | 0.01 | ND | 0.10% | 0.10% | ND |
| | 1.14 | ≤0.15% | ND | ND | ND | 0.02% | ND |
| | 1.33 | ≤0.15% | ND | 0.05% | 0.10% | 0.10% | 0.05% |
| | 1.35 | ≤0.15% | 0.01% | ND | ND | ND | ND |
| | 2.46 | ≤0.15% | ND | 0.02% | ND | ND | ND |
| | 3.28 | ≤0.15% | ND | 0.04% | 0.03% | ND | ND |

| | | | | | | |
|---|---|---|---|---|---|---|
| Total Impurities | ≤5.0% | 0.13% | 0.28% | 0.62% | 0.80% | 0.22% |

ND denotes not detected

As shown in Tables 2-5, Compositions X and 2 provided stable cannabidiol at accelerated storage conditions.

### Example 3

### Pharmacokinetic Study in Dogs

TABLE 6
Composition A

| % w/w | A |
|---|---|
| Cannabidiol | 10.53 |
| Vitamin E | 0.2 |
| Saccharin | 0.025 |
| Strawbeny Flavor | 0.3 |
| Miglyol ® 812 (C8/C10 medium | 88.945 |
| chain triglycerides) | |

### Methods

In-vivo bioavailability and pharmacokinetics of Composition A, from Table 6, and Composition 1, from Table 1, was evaluated in Beagle dogs. Specifically, two sets of five male Beagle dogs weighing from about 5 to about 11 kilograms were fasted overnight before dosing. Each Beagle dog was then administered 200 milligrams of cannabidiol in the form of Composition A or Composition 1. Blood samples were collected at 0, 15 and 30 minutes and 1, 2, 3, 4, 8, 12, 24, 48, 72 and 96 hours after dosing.

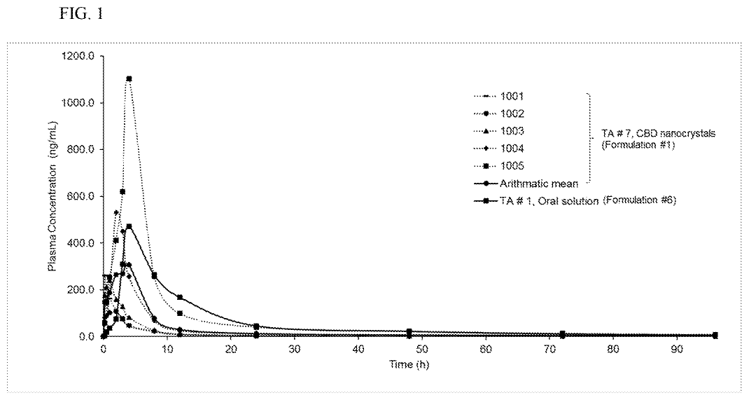

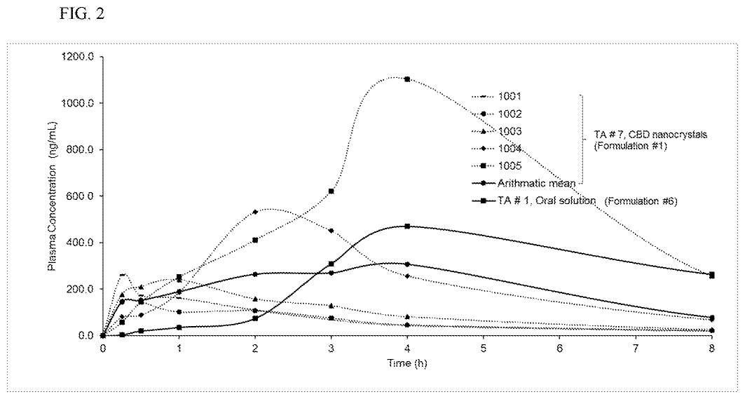

The following pharmacokinetic parameters were calculated: peak concentration in plasma (“Cₘₐₓ”), time to peak concentration (“Tₘₐₓ”) and area under the concentration-time curve (“AUC”). Results of this study can be seen in Table 7, below and in FIGS. 1 and 2.

TABLE 7
Pharmacokinetic parameters

| PK Parameters | | Formulation A | Formulation 1 |
|---|---|---|---|
| Cmax (ng/mL) | | 470.36 ± 297.36 | 319.85 ± 481.09 |
| Tmax (h) | | 4 | 4 |
| AUC | 0-1 h | 17.6 ± 7.5 | 143.2 ± 53.4 |
| | 0-4 h | 654.7 ± 337.7 | 942.6 ± 786 |
| | 0-12 h | 2987.5 ± 1917.9 | 1972.8 ± 2258.7 |
| | 0-24 h | 4268.7 ± 3179.1 | 2232.1 ± 2577.5 |
| | 0-48 h | 5081.0 ± 3776.3 | 2232.1 ± 2577.5 |
| | 0-96 h | 5705 ± 4229.7 | 2709.5 ± 3161.7 |
| Relative | 0-1 h | 100 | 812.7 |
| bio- | 0-4 h | 100 | 144 |
| availability | 0-12 h | 100 | 66 |
| % | 0-24 h | 100 | 52.3 |
| | 0-48 h | 100 | 48.7 |
| | 0-96 h | 100 | 47.5 |

### Results

As seen in FIGS. 1 and 2 and Table 7, Composition 1 provides a higher plasma concentration at both 1 and 4 hours after administration than Composition A.

Benefits, other advantages, and solutions to problems have been described herein with regard to specific embodiments. Furthermore, the connecting lines shown in various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the inventions. The scope of the inventions is accordingly to be limited by nothing other than the appended claims, in which reference to an element in the singular is not intended to mean “one and only one” unless explicitly so stated. but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in a single embodiment; for example, A and B, A and C, B and C. or A and B and C. Different cross-hatching is used throughout the figures to denote different parts but not necessarily to denote the same or different materials.

Systems, methods and apparatus are provided herein. In the detailed description herein, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further. when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant arts) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U S C. 112(f), unless the element is expressly recited using the phrase “means for.” As used herein, the terms “comprises”, “comprising”, or any other variation thereof, are intended to cover a non-exclusive inclusion. such that a process, method, article or apparatus that comprises a list of elements does not include only those elements but may include other elements not expressly listed or inherent to such process, method or article, or apparatus.

1. A stable nanocrystal composition comprising from about 1% to about 40% w/w cannabidiol, one or more lipids selected from stearoyl polyoxyl-32 glycerides, polyethylene glycol monostearate, glyceryl dibehenate, glyceryl distearate, propylene glycol monocaprylate, oleoyl polyoxyl-6 glycerides and linoleoyl polyoxyl-6 glycerides and one or more stabilizers selected from hydroxypropyl cellulose, hydroxypropyl methyl cellulose, polyvinyl pyrrolidine and a poloxamer, wherein w/w denotes weight by total weight of composition.

2. The composition of claim 1, further comprising one or more surfactants selected from cetyl trimethylammonium bromide (CTAB) and cetyl trimethylammonium chloride (CTAC), ammonium lauryl sulfate, sodium lauryl sulfate, polysorbate 20, polysorbate 40, polysorbate 60, polysorbate 80, sorbitan monolaurate, sorbitan monopalmitate, sorbitan monostearate, and sorbitan monooleate, poloxamer 188 and poloxamer 407.

3. The composition of claim 1, further comprising one or more cosolvents selected from water, propylene glycol and ethanol.

4. The composition of claim 1, further comprising one or more antioxidants selected from pegylated alpha-tocopherol isomer of vitamin E, alpha-tocopherol, ascorbic acid, ascorbyl palmitate, butylated hydroxyanisole and butylated hydroxytoluene.

5. The composition of claim 1, further comprising one or more preservatives selected from meta-cresol, benzalkonium chloride, methyl paraben and propyl paraben.

6. The composition of claim 1, further comprising disodium edetate.

7. The composition of claim 1, wherein the one or more surfactants is selected from the group of polysorbate 80, sorbitan monooleate and poloxamer 188, wherein the ratio of polysorbate 80 or sorbitan monooleate to poloxamer 188 is about 2:1.

8. The composition of claim 1, wherein the one or more stabilizers is hydroxypropyl cellulose L and wherein the ratio of cannabidiol to hydroxypropyl cellulose L is about 2:1.

9. The composition of claim 1, wherein the composition forms particles having a mean particle size from about 100 to about 1000 nanometers.

10. The composition of claim 1, wherein the composition forms particles having a mean particle size from about 200 to about 500 nanometers.

11. The composition of claim 1, wherein the composition forms particles having a mean particle size from about 250 to about 300 nanometers.

12. A nanocrystal composition comprising from about 5% to about 20% w/w cannabidiol;
from about 0.1% to about 5% w/w of one or more lipids selected from stearoyl polyoxyl-32 glycerides, polyethylene glycol monostearate, glyceryl dibehenate, glyceryl distearate, propylene glycol monocaprylate, oleoyl polyoxyl-6 glycerides and linoleoyl polyoxyl-6 glycerides; and
from about 1% to about 10% w/w of one or more stabilizers selected from hydroxypropyl cellulose, hydroxypropyl methyl cellulose, polyvinyl pyrrolidine and a poloxamer,
wherein w/w denotes weight by total weight of composition.

13. The composition of claim 12, further comprising:
from about 1% to about 10% w/w of one or more surfactants selected from polysorbate 20, polysorbate 40, polysorbate 60, polysorbate 80, sorbitan monolaurate, sorbitan monopalmitate, sorbitan monostearate, and sorbitan monooleate, poloxamer 188 and poloxamer 407; and
from about 50% to about 90% w/w of one or more cosolvents selected from water, propylene glycol and ethanol.

14. The composition of claim 9, further comprising from about 0.1% to about 5% w/w of one or more preservatives selected from pegylated alpha-tocopherol isomer of vitamin, alpha-tocopherol, ascorbic acid, ascorbyl palmitate, methyl paraben and propyl paraben.

15. The composition of claim 9, further comprising from about 0.01% to about 0.5% w/w disodium edetate.

16. (canceled)

17. A process of producing a nanoparticle composition comprising the steps of:
adding ascorbic acid or vitamin E pegylated, polysorbate 80, poloxamer 188, hydroxypropyl cellulose L and optionally, disodium edetate to water were while stirring to create an aqueous phase;
adding cannabidiol to ethanol while stirring to create an alcohol phase;
adding the alcohol phase to the aqueous phase dropwise while spinning in a homogenizer at from about 13,000 to about 17,000 revolutions per minute for 5 minutes to form a coarse mixture;
placing the coarse mixture in a pressure homogenizer for from about 5 to about 10 cycles at from about 10,000 to about 20,000 pounds per square inch to create a homogenous mixture; and
allowing the homogenous mixture to reach room temperature, wherein the process provides a particle size range from about 200 to about 500 nanometers.

18. A method of treating a disease selected from Prader-Willi syndrome, obesity, graft versus host disease, gelastic seizures/hypothalamic hamartoma, neonatal seizures, dystonia, central pain syndromes, phantom limb pain, multiple sclerosis, traumatic brain injury, radiation therapy, acute graft versus host disease, chronic graft versus host disease, T-cell autoimmune disorders, colitis, Dravet Syndrome, Lennox Gastaut Syndrome, mycolonic seizures, juvenile mycolonic epilepsy, refractory epilepsy, childhood absence epilepsy, schizophrenia, juvenile spasms, West syndrome, infantile spasms, refractory infantile spasms, tuberous sclerosis complex, brain tumors, neuropathic pain, cannabis use disorder, post-traumatic stress disorder, anxiety, early psychosis, Alzheimer's Disease, autism, acne, Parkinson's disease, social anxiety disorder, depression, diabetic retinopathy, diabetic nephropathy, diabetic neuropathy, ischemic injury of heart, ischemic injury of brain, chronic pain syndrome, and rheumatoid arthritis comprising administering a composition of claim 1 to a subject in need thereof.

19. A method of treating withdrawal symptoms comprising administering a composition of claim 1 to a subject in need thereof, wherein the withdrawal symptoms are caused by the subject reducing or quitting use of an opioid, cocaine, heroin, an amphetamine or nicotine.
